(19) United States
(12) Patent Application Publication
(10) Pub. No.: US 2018/0228698 A1
(43) Pub. Date: Aug. 16, 2018

(54) Double Balloon Floating Feeding Jejunostomy Tube

(71) Applicant: New York University, New York, NY (US)

(72) Inventor: George S. Ferzli, New York, NY (US)

(21) Appl. No.: 15/894,453

(22) Filed: Feb. 12, 2018

Related U.S. Application Data
(60) Provisional application No. 62/458,333, filed on Feb. 13, 2017.

(51) Int. Cl. A61J 15/00

(52) U.S. Cl. CPC A61J 15/0049; A61M 25/10182; A61J 15/0069

(57) ABSTRACT

A jejunostomy feeding device includes an elongate flexible member extending from a proximal end to a distal end and including a feeding lumen extending therethrough. The device also includes a first balloon attached to a distal portion of the flexible member. In addition, the device includes a second balloon attached to the flexible member proximally of the first balloon, the second balloon including a groove extending about at least a portion of a circumference thereof to anchor the second balloon within target anchoring tissue upon inflation thereof, wherein the first balloon and the second balloon are separated by a first distance selected so that when the second balloon is positioned at a target site within a target body lumen, the second balloon anchors the first balloon within the target body lumen positioned so that the first balloon presses against an inner wall of the body lumen to seal an entry point at which the flexible member enters the body lumen.

### PRIORITY CLAIM

The present disclosure claims priority to U.S. Provisional Patent Application Ser. No. 62/458,333 filed Feb. 13, 2017; the disclosure of which is incorporated herewith by reference.

### BACKGROUND

Placement of jejunostomy tubes for nutritional support in patients undergoing upper digestive tract surgery is common practice. In nutritionally depleted cancer patients requiring adjuvant chemotherapy and its associated side effects including food intolerance, their role is even more pronounced. Moreover, enteral feeding has been shown to be superior to intravenous feeding as it maintains the integrity of the mucosa with trophic factors preventing bacterial translocation and enhancing immunity. However, placement of jejunostomy feeding tubes is not without complications. Tube dislodgment, small bowel obstruction, and surgical site infection are among the complications and may occur. Despite advances in surgical technique and equipment, percutaneous needle catheters and laparoscopy, the complication rate has remained substantially consistent. All current methods, whether open, laparoscopic, or percutaneous are based on fixing the small bowel to the abdominal wall and cannulation of the small bowel at that level. Considering that the small bowel is not naturally a fixed organ, and that many of the complications such as obstruction and tube dislodgment are a direct cause of this fixation, a method that does not fix the bowel to the anterior abdominal wall is proposed.

### SUMMARY

In accordance with the foregoing objectives and others, one embodiment of the present invention provides jejunostomy feeding devices and methods for placement thereof.

In one aspect, a jejunostomy feeding device is provided. The device includes an elongate flexible member extending from a proximal end to a distal end defining a feeding lumen extending therethrough and including a first balloon attached to a distal portion of the flexible member and a second balloon attached to the flexible member proximally of the first balloon, the second balloon including a groove extending about at least a portion of a circumference thereof to anchor the second balloon within target anchoring tissue upon inflation thereof, wherein the first balloon and the second balloon are separated by a first distance selected so that when the second balloon is positioned at a target site within a target body lumen, the second balloon anchors the first balloon within the target body lumen positioned so that the first balloon presses against an inner wall of the body lumen to seal an entry point at which the flexible member enters the body lumen. In one embodiment, the first balloon is inflatable via a first inflation lumen extending through the flexible member and the second balloon is inflatable via a second inflation lumen extending through the flexible member. In an embodiment, the first and second inflation lumens extend from respective proximal openings formed in a proximal portion of the flexible member to openings within the first and second balloons, respectively. In another embodiment, the first distance is between 3 and 5 cm. In an embodiment, each of the proximal openings are in open communication with an access port. In another embodiment, the first balloon is positioned approximately 5 cm from the distal end of the flexible member. In a further embodiment, the flexible member includes a plurality of openings along a distal end thereof in communication with the feeding lumen. In a further embodiment, the device further includes a weighted distal tip coupled to the distal end of the flexible member.

In one aspect, a method for placement of a jejunostomy feeding device is provided. The method includes advancing an elongate flexible member into a small bowel along a path parallel to a known blood vessel, the flexible member extending from a proximal end to a distal end and including a feeding lumen extending therethrough, the flexible member having a length selected so that, when the distal end is in the small bowel, the proximal end is outside the body, inserting a distal end of the flexible member into the small bowel through a mesenteric side thereof until a distal portion of the flexible member extends within the small bowel, the distal portion of the flexible member including a first balloon attached thereto, inflating the first balloon within the small bowel, and inflating a second balloon attached to the flexible member proximally of the first balloon within the mesentery, the second balloon including an anchoring element to anchor the flexible member at a target location within the mesentery upon inflation thereof. In one embodiment, the anchoring element is a groove extending about at least a portion of a circumference of the second balloon. In an embodiment, the method further includes inserting a needle into the mesentery tangentially at an angle of substantially 0 degrees towards the anti-mesenteric side of the small bowel to determine an insertion path along which the flexible member is to be advanced into the small bowel. In another embodiment, the method further includes inserting a guide wire through the needle and into the small bowel along the insertion path. In a further embodiment, the first balloon is inflatable via a first inflation lumen. In another embodiment, the second balloon is inflatable via a second inflation lumen.

In one aspect, method for placement of a feeding device is provided. The method includes advancing an elongate flexible member into a small bowel along a path parallel to a known blood vessel, the flexible member extending from a proximal end to a distal end and including a feeding lumen extending therethrough. The method also includes inserting a distal end of the flexible member into the small bowel through a mesenteric side thereof until a distal portion of the flexible member extends within the small bowel, the distal portion of the flexible member including a first balloon attached thereto. The method further includes inflating a second balloon attached to the flexible member proximally of the first balloon within the mesentery to fix the first balloon in a target location. In one embodiment, the second balloon includes an anchoring element to anchor the flexible member at a target location within the mesentery upon inflation thereof. In an embodiment, the method includes inflating the first balloon so that the first balloon presses against an inner wall of the body lumen to seal an entry point at which the flexible member enters the body lumen. In another embodiment, the method includes inserting a needle into the mesentery tangentially at an angle of substantially 0 degrees towards the anti-mesenteric side of the small bowel to determine an insertion path along which the flexible member is to be advanced into the small bowel. In a further embodiment, the method further includes inserting a guide wire through the needle and into the small bowel along the insertion path. In a further embodiment, the first balloon is inflatable via a first inflation lumen and the second balloon is inflatable via a second inflation lumen.

### BRIEF DESCRIPTION OF THE DRAWINGS

FIG. 1 shows a perspective view of a feeding device according to an exemplary embodiment of the present disclosure;

FIG. 2 shows a cross-sectional view of the longitudinal member of the device of FIG. 1;

FIG. 3 shows a perspective view of an exemplary method for placing the feeding device of FIG. 1;

FIG. 4 shows a second perspective view of an exemplary method for placing the feeding device of FIG. 1;

FIG. 5 shows a third perspective view of an exemplary method for placing the feeding device of FIG. 1; and

FIG. 6 shows a fourth perspective view of an exemplary method for placing the feeding device of FIG. 1.

### DETAILED DESCRIPTION

The present disclosure may be further understood with reference to the following description and the appended drawings, wherein like elements are referred to with the same reference numerals. The present embodiments relate generally to jejunostomy feeding devices and methods of placement thereof. In particular, the present embodiments relate to devices and method of anchoring a jejunostomy feeding catheter within the mesentery. However, the present embodiments may be employed with any of a plurality of treatment procedures involving catheters and related devices. As used in this application, the terms proximal and distal refer to a direction along the feeding catheter with a first end of the catheter being identified as the proximal end and a second end of the catheter being identified as distal.

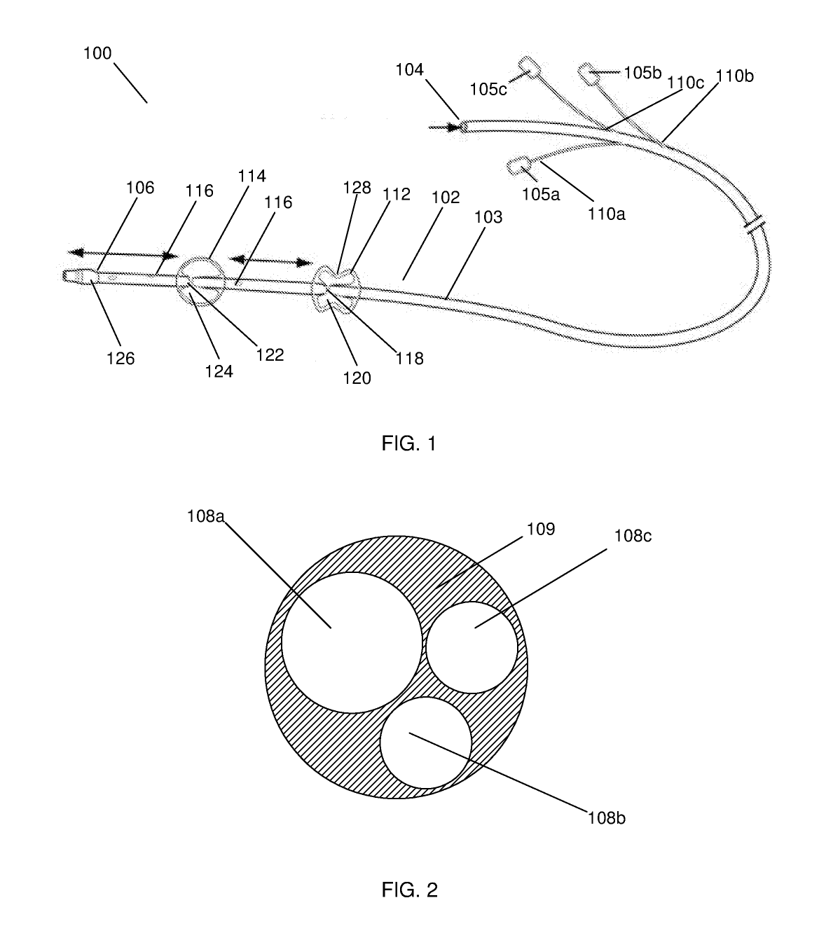

As shown in FIGS. 1-2, a jejunostomy feeding device 100 according to a first exemplary embodiment comprises a flexible member 102, a first mesenteric balloon 112 and a second intralumenal balloon 114. As would be understood by those skilled in the art, the device 100 is preferably sufficiently flexible to pass through a tortuous path, for example, within a natural body lumen without undue trauma to tissue along and adjacent to the lumen or damage to the device 100. For example, the device 100 may have a flexibility sufficient to permit the device 100 to be slidably inserted along a path parallel to a known blood vessel and to pass through any bending radii that this path might require. The flexible member 102 may be formed as a catheter and has a body 110 formed of a flexible, biocompatible material such as silicone, polyurethanes (PU), polytetrafluroethylene (PTFE), polyisobutylene polyurethane (PIB-PUR), poly(styrene-block-isobutylene-block-styrene) (SIBS), or any other suitable biocompatible flexible material. According to the exemplary embodiment, the flexible member 102 may be formed, for example, as a kink-resistant tubing having an outer diameter of 3.3 mm (10 FR). The tube may vary from, for example, 1.32 mm (4 FR) in children to 5.95 mm (18 FR) in adults and obese. The length of the flexible member 102 may be approximately 40 cm (in children) to 110 cm (in adults). The flexible member 102 of the exemplary embodiment extends between a proximal end 104 which, in use, remains outside the body accessible to the user, and a distal end 106 extending into the lumen of a target organ (i.e., jejunum). The flexible member 102 includes a plurality of lumens 108 extending from the proximal end 104 to a distal portion. The proximal end of the flexible member 102 includes one or more ports 105 for passing desired fluids, medicines, nutrition and/or apparatus as desired into or out of the patient. In an exemplary embodiment, the flexible member 102 of this embodiment includes a main flexible member flushing access port 105a, a mesenteric balloon insufflation port 105b and an intralumenal balloon insufflation port 105c. However, it will be understood that any number of ports may be employed for passage of fluids, solids, etc. In one embodiment, the access ports 105 may include a valve (not shown) configured to selectively control fluid flow through one or more of the lumens 108 of the flexible member. Suitable valves (not shown) include, for example, one-way and two-way valves. In some examples, the ports 105a, 105b, 105c may each independently include a luer lock connector optionally with a stopcock and/or check valve. Valves (not shown) may assist in fluid regulation to/from the patient. For example, it may be preferable that the mesenteric balloon 112 and intralumenal balloon 114 include a one-way check valve which can assist with inflation and/or deflation of the balloon during insertion and removal, as well as maintaining a preferred level of inflation. In particular, lumen 108b which is in communication with the mesenteric balloon 112, may terminate at its proximal end 110b into a first luer lock connectable to an inflation pump and/or a syringe, the first luer lock may optionally comprise a stopcock and/or a check valve. Similarly, lumen 108c which is in communication with the intralumenal balloon 114, may terminate at its proximal end 110c into a second luer lock connectable to an inflation pump and/or a syringe, the second luer lock may optionally comprise a stopcock and/or a check valve. In one particular example, the mesenteric balloon insufflation port 105b and the intralumenal balloon insufflation port 105c may each independently comprise a luer lock connectable to an inflation pump or a syringe, where the luer lock may each optionally comprise a stopcock and/or a check valve.

The ports 105a, 105b, 105c are each coupled to a corresponding lumen 108a, 108b, 108c, respectively, extending down a portion of the length of the flexible member 102. In an exemplary embodiment shown in FIG. 1, the flexible member 102 includes three lumens 108a, 108b, 108c having proximal ends 110a, 110b, 110c in communication with at least one of the ports 105. In the exemplary embodiment of FIG. 1, main flexible member flushing port 105a is in communication with main flexible member lumen 108a, mesenteric balloon insufflation port 105b is in communication with lumen 108b and intralumenal balloon insufflation port 105c is in communication with lumen 108c. The lumens 108 are separated from each other but extend parallel to one another, e.g., are separated by longitudinally extending walls 109. Each of the lumens 108 has a predetermined length and diameter selected to accommodate appropriate fluid/gas access as would be understood by those skilled in the art. In an exemplary embodiment, shown in FIG. 2, the main flexible member lumen 108a has a larger diameter that lumens 108c and 108b to allow for passage of a greater amount of fluids or fluids including solids therein. In this embodiment, Lumen 108a may have a diameter of, for example, 3.2 mm (9.6 FR) while lumens 108b and 108c have a diameter of, for example, 1 mm (3 FR). It will be understood that the lumens may take any shape, size or diameter necessary.

Each of the lumens 108 extends to a corresponding distal end including at least one exit port. In an exemplary embodiment, main lumen 108a extends the length of the flexible member 102 to the distal end 106. The lumen 108a has an opening at the distal end 106 to allow flow of fluid into the patient's jejunum. Thus, fluid communication is established from the main flexible member flushing access port 105a through the lumen 108a to the exterior of the distal end of the flexible member 102. In an exemplary embodiment, flexible member 102 also includes a plurality of openings 116 extending through a wall of the distal portion of the flexible member 102 to open the lumen 108a to the exterior of the member 102. The openings 116 in this embodiment are distributed about the circumference of the member 102 in a substantially helical pattern and, in this embodiment, are distributed evenly around a circumference of the flexible member 102. It is noted however, that openings 116 may be formed in any pattern or arrangement on the flexible member 102 without deviating from the scope of the present embodiments. For example, in an alternate embodiment (not shown), the openings 116 may be situated around only a portion of the circumference of the flexible member 102. The diameter of each of the openings 116 is selected to allow fluid to flow therethrough. For example, the openings 116 may have a diameter of 3.3 mm.

The lumen 108b extends from a proximal end 110b in communication with the mesenteric balloon insufflation port 105b to a distal opening 118 in communication with an annular space 120 of the mesenteric balloon 112, as will be described in further detail below. The distal opening 118 allows fluid/gas to flow into and out of the annular space 120 to inflate or deflate the balloon 112. Similarly, lumen 108c extends from proximal end 110c in communication with the intralumenal balloon insufflation port 105c to a distal opening 122 at a distal end in communication with an annular space 124 of the intralumenal balloon 114. The distal opening 122 allows for fluid/gas to flow into and out of the annular space 124 to inflate or deflate the balloon 114. Each of the distal openings 118, 122 may be formed as a single opening or a plurality of openings so long as a fluid tight seal is formed between the lumens 108b, 108c and the annular spaces 120, 124 of their respective balloons 112, 114.

The distal end 106 of the flexible member 102 of this embodiment is adapted to terminate within the jejunum. However, the path and positioning of the distal portion of the flexible member 102 including its distal end 106, will vary from patient to patient. For example, the placement may depend to some degree on the method of insertion or placement. To assist in the placement of the device 100, the flexible member 102 may include a weighted distal tip 126 attached to the distal end 106. The distal tip 126 may be formed as a substantially rigid tip to aid in placement of the distal end 106. Furthermore, the distal tip 126 may resist collapse of the end 106 of the flexible member 102, preventing or reducing the likelihood of blockage or clogging of the flexible member 102. In an exemplary embodiment, the tip 126 is rounded to prevent abrasion to surrounding tissue or blood vessels.

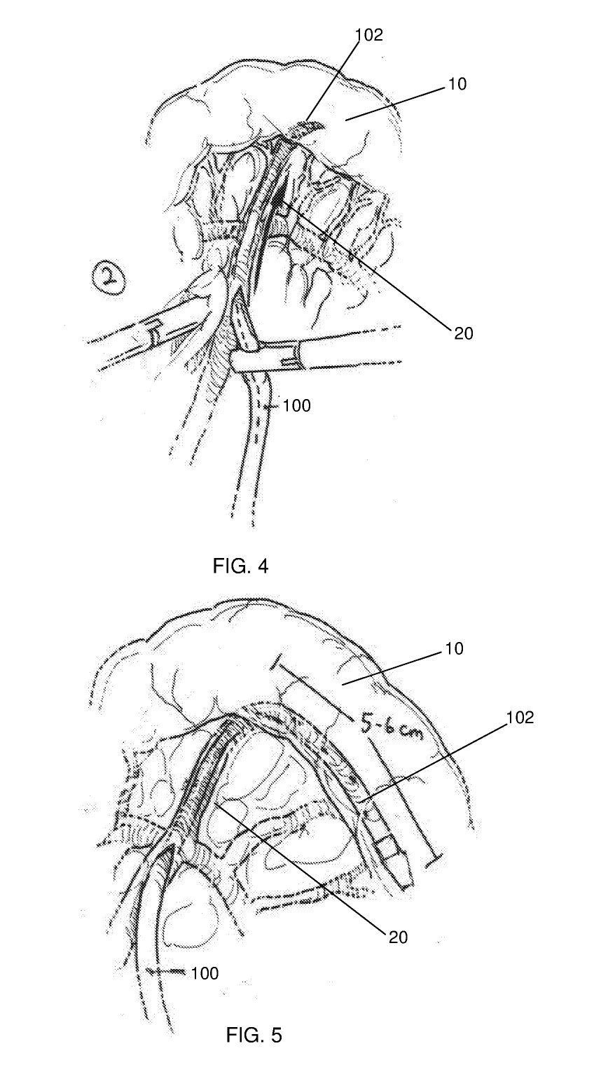

The mesenteric balloon 112 is movable between a deflated state and an inflated state and is used to assist with the positioning of the flexible member 102. The balloon 112 may be formed as a substantially cylindrical sleeve overlying and extending about a distal portion of the flexible member 102. The balloon 112 may be attached to the flexible member 102 at, for example, proximal and distal ends of the balloon 112, or, in another example, may be attached to the flexible member 102 along an entirety of the length of the balloon 112. In another embodiment, the balloon 112 may be unitarily formed with the flexible member 102 or secured to the interior of the flexible member 102. The balloon 112 may be formed of rubber or any other suitable elastomeric material and is preferably sufficiently thin so that it is easily movable through a lumen or a target path alongside blood vessels in a deflated state. In an inflated state, the balloon 112 has a volume of between 0.5 ml and 1.5 ml. Once inflated, the mesenteric balloon 112 serves to keep the flexible member 102 in place such that can not easily be withdrawn or dislodged from the target position at which it was inflated within the mesentery. That is, the balloon 112 generally inflates or expands so that the flexible member 102 is retained in place until the balloon 12 is deflated and withdrawn from the target position. When the balloon 112 is in the deflated state as shown in FIGS. 5-4, the flexible member 102 is easily movable into and out of the body and along the target insertion path. The mesenteric balloon 112 is positioned at a location on the flexible member 102 selected so that, when the balloon 112 is inflated at the target position within the mesentery 30, the distal end 106 of the flexible member 102 extends into the target organ lumen of the patient (i.e., the jejunum). In an exemplary embodiment, the balloon 112 may be located between 8 and 10 cm from the distal end 106 of the flexible member 102 and approximately 3-5 cm proximally of the intralumenal balloon 114. However, it will be understood that the balloon 112 may be positioned anywhere on the flexible member 102 depending on the patient's anatomy, provided that it allows the desired retention to occur with the distal end 106 positioned as desired within the target organ. In an exemplary embodiment, the balloon 112 includes an anchoring element to aid in fixation of the balloon 112 within the mesentery. For example, as can be seen in FIG. 1, the balloon 112 includes a groove 128 extending about a medial portion thereof. In one embodiment, the groove 128 extends about the entire circumference of the balloon 112. In another embodiment, the groove 128 may extend about only a portion of the circumference of the balloon.

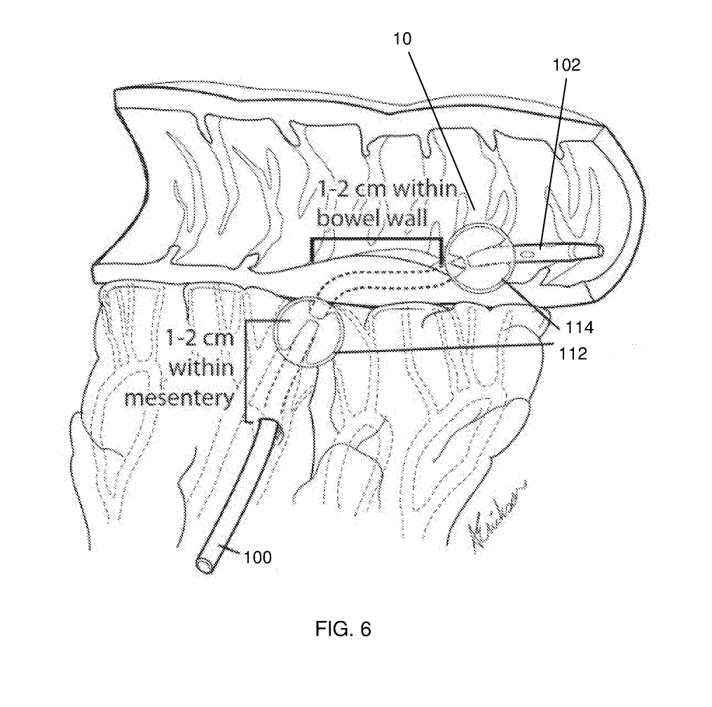

Similarly to the expandable mesenteric balloon 112, the intralumenal balloon 114 is movable between a deflated state and an inflated state and is used to assist with the positioning of the flexible member 102 within the lumen 10 of a target organ (e.g., the small bowel 10). The balloon 114 may be formed as a substantially spherical sleeve overlying and extending about the flexible member 102. The balloon 114 may be attached to the flexible member 102 at, for example, proximal and distal ends of the balloon 114, or, in another example, the balloon 114 may be attached to the flexible member 102 along an entire length of the balloon 114. In another embodiment, the balloon 114 may be unitarily formed with the flexible member 102 or secured to the interior of the flexible member 102. The balloon 114 may be formed of rubber or any other suitable material and is preferably sufficiently thin so that, in the deflated state, it is easily movable through tissue, through a body lumen or any other target path (e.g., alongside blood vessels 20) to the target site in the body. In the inflated state, the balloon 114 has a volume of between 0.5 ml and 1.5 ml. Once inflated, the intralumenal balloon 114 serves to keep the flexible member 102 in place such that it is not readily removed or dislodged from the target site (e.g., within the jejunum). That is, the balloon 114 generally inflates or expands so that the flexible member 102 is retained in a target position until the balloon 114 is deflated and withdrawn therefrom. The intralumenal balloon 114, when inflated, is also positioned to press against the inner wall of the small bowel lumen 10 to seal the entry point at which the flexible member 102 entered the lumen. Such sealing prevents leakage of gastric contents via the tunnel formed by the flexible member 102. When the balloon 114 is in the deflated state as shown in FIGS. 4-5, the flexible member 102 is easily movable into and out of the body and along the target insertion path. The intralumenal balloon 114 is positioned on a distal portion of the flexible member 102, distally of mesenteric balloon 112, at a location selected so that, when the balloon 114 is in a sealing position at the point of entry of the flexible member 102 into the target organ (e.g., the jejunum), the distal end 106 of the flexible member 102 is in a target location within the target organ, as can be seen in FIG. 6. In an exemplary embodiment, the balloon 114 may be located approximately 5 cm proximally from the distal end 106 of the flexible member 102. However, it will be understood that the balloon 114 may be positioned anywhere on the flexible member 102 depending on the patient anatomy and desired placement of the distal end 106, provided that it allows the desired retention and sealing within the lumen of the small bowel to occur.

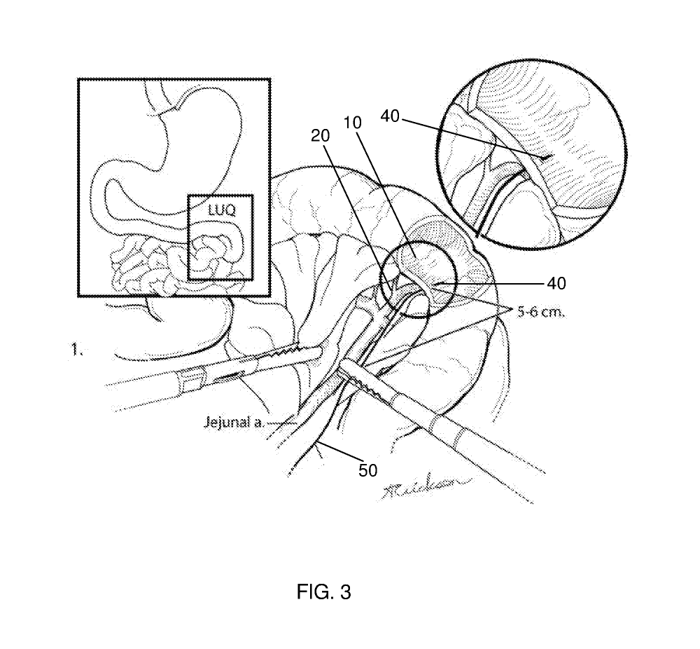

An exemplary method for placement of device 100 can be seen in FIGS. 3-6. Initially, a target loop of the jejunum approximately 30 cm from the ligament of the trietz is identified. A mesenteric tunnel is established to provide access into the small bowel lumen 10 from the mesenteric side. The tunnel may be between 3 and 5 cm in length and can be formed by insertion of a needle 40 into the mesentery 30 tangentially at, for example, 0 degrees to 15 degrees and aiming toward an anti-mesenteric side of the small bowel 10. To avoid inadvertent cannulation of blood vessels 20, the needle 40 may be inserted along and anterior to a path of the vessels 20. If preferred, confirmation of the intralumenal location of the needle 40 can be done by inspection, palpation, aspiration of enteric contents into a fluid filled syringe or by injecting fluid into the bowel lumen 10. A guide wire 50 may then be introduced through the needle and oriented into the distal small bowel 10. The needle 40 is removed and the tract is dilated over the guide wire 50 with, for example, a 5F sheath. The dilators are removed and the device 100 is inserted distally into the small bowel 110 over the guidewire. The intralumenal position of the flexible device 102 is confirmed by clamping the small bowel 10 proximally and distally and distending the small bowel 10 (e.g., through the introduction of air or water therein).

After the flexible device 102 is in a target position, the device 100 is fixed in this position by inflating the mesenteric balloon 112 through insertion of fluid, such as water, into the mesenteric balloon insufflation port 105b. After the balloon 112 has been inflated, the groove 118 anchors the balloon 112 in the mesentery. The intralumenal balloon 114 may then be inflated through introduction of fluid, such as water, into the intralumenal insufflation port 105c. It will be understood that the intralumenal balloon 114 may be inflated before, after, or concurrently with inflation of the mesenteric balloon 112. When the balloon 114 is inflated, it presses against an inner wall of the small bowel 10 sealing the entry point thereto and preventing leakage of gastric contents via the tunnel formed by the needle 40. Once both balloons 112, 114 have been inflated in their target positions, the small bowel 10 is dropped back into the peritoneal cavity and the flexible member 102 is brought through the fascia. The device 100 is then fixed to the patient at the skin level to be clamped or accessed as needed.

It will be apparent to those skilled in the art that various modifications and variations may be made to the structure and methodology of the present invention without departing from the spirit or scope of the invention. Thus, the present invention covers all modifications and variations so long as they come within the scope of the appended claims and their equivalents.

What is claimed is:

1. A jejunostomy feeding device, comprising:
an elongate flexible member extending from a proximal end to a distal end and including a feeding lumen extending therethrough;
a first balloon attached to a distal portion of the flexible member; and
a second balloon attached to the flexible member proximally of the first balloon, the second balloon including a groove extending about at least a portion of a circumference thereof to anchor the second balloon within target anchoring tissue upon inflation thereof;
wherein the first balloon and the second balloon are separated by a first distance selected so that when the second balloon is positioned at a target site within a target body lumen, the second balloon anchors the first balloon within the target body lumen positioned so that the first balloon presses against an inner wall of the body lumen to seal an entry point at which the flexible member enters the body lumen.

2. The device of claim 1, wherein the first balloon is inflatable via a first inflation lumen extending through the flexible member and the second balloon is inflatable via a second inflation lumen extending through the flexible member.

3. The device of claim 1, wherein the first and second inflation lumens extend from respective proximal openings formed in a proximal portion of the flexible member to openings within the first and second balloons, respectively.

4. The device of claim 1, wherein the first distance is between 3 and 5 cm.

5. The device of claim 3, wherein each of the proximal openings are in open communication with an access port.

6. The device of claim 5, wherein each of the access ports comprises a luer lock connector configured to connect to an inflation pump or a syringe.

7. The device of claim 6, wherein the luer lock connector comprises at least one of a stopcock and a check valve.

8. The device of claim 1, wherein the first balloon is positioned approximately 5 cm from the distal end of the flexible member.

9. The device of claim 1, wherein the flexible member includes a plurality of openings along a distal end thereof in communication with the feeding lumen.

10. The device of claim 1, further comprising a weighted distal tip coupled to the distal end of the flexible member.

11. A method for placement of a jejunostomy feeding device, comprising:
advancing an elongate flexible member into a small bowel along a path parallel to a known blood vessel, the flexible member extending from a proximal end to a distal end and including a feeding lumen extending therethrough, the flexible member having a length selected so that, when the distal end is in the small bowel, the proximal end is outside the body;
inserting a distal end of the flexible member into the small bowel through a mesenteric side thereof until a distal portion of the flexible member extends within the small bowel, the distal portion of the flexible member including a first balloon attached thereto;
inflating the first balloon within the small bowel; and
inflating a second balloon attached to the flexible member proximally of the first balloon within the mesentery, the second balloon including an anchoring element to anchor the flexible member at a target location within the mesentery upon inflation thereof.

12. The method of claim 11, wherein the anchoring element is a groove extending about at least a portion of a circumference of the second balloon.

13. The method of claim 11, further comprising:
inserting a needle into the mesentery tangentially at an angle of substantially 0 degrees relative to the mesentery towards the anti-mesenteric side of the small bowel to determine an insertion path along which the flexible member is to be advanced into the small bowel.

14. The method of claim 13, further comprising:
inserting a guide wire through the needle and into the small bowel along the insertion path.

15. The method of claim 11 wherein the first balloon is inflatable via a first inflation lumen.

16. The method of claim 11, wherein the second balloon is inflatable via a second inflation lumen.

17. A method for placement of a feeding device, comprising:
advancing an elongate flexible member into a small bowel along a path parallel to a known blood vessel, the flexible member extending from a proximal end to a distal end and including a feeding lumen extending therethrough;
inserting a distal end of the flexible member into the small bowel through a mesenteric side thereof until a distal portion of the flexible member extends within the small bowel, the distal portion of the flexible member including a first balloon attached thereto;
inflating a second balloon attached to the flexible member proximally of the first balloon within the mesentery to fix the first balloon in a target location.

18. The method of claim 17, wherein the second balloon includes an anchoring element to anchor the flexible member at a target location within the mesentery upon inflation thereof

19. The method of claim 17, further comprising:
inflating the first balloon so that the first balloon presses against an inner wall of the body lumen to seal an entry point at which the flexible member enters the body lumen.

20. The method of claim 17, further comprising:
inserting a needle into the mesentery tangentially at an angle of substantially 0 degrees relative to the mesentery towards the anti-mesenteric side of the small bowel to determine an insertion path along which the flexible member is to be advanced into the small bowel.

21. The method of claim 17, further comprising:
inserting a guide wire through the needle and into the small bowel along the insertion path.

22. The method of claim 17, wherein the first balloon is inflatable via a first inflation lumen and the second balloon is inflatable via a second inflation lumen.
